(19) United States
(12) Patent Application Publication
(10) Pub. No.: US 2022/0175774 A1
(43) Pub. Date: Jun. 9, 2022

(54) Bioavailable Oral Dosage Form Of Tyrosine-Kinase Inhibitor

(71) Applicant: Alembic Pharmaceuticals Limited, Vadodara (IN)

(72) Inventors: Barada Prasanna Sahoo, Hyderabad (IN); Naveen Kumar Midathanapally, Hyderabad (IN); Rahul Sudhakar Dabre, Hyderabad (IN); Pankaj Kumar Khapra, Vadodara (IN)

(73) Assignee: Alembic Pharmaceuticals Limited, Vadodara (IN)

(21) Appl. No.: 17/542,738

(22) Filed: Dec. 6, 2021

(30) Foreign Application Priority Data
Dec. 7, 2020 (IN) 202021053103

(51) Int. Cl. A61K 31/506; A61K 9/16; A61K 9/20; A61K 9/28

(52) U.S. Cl. CPC A61K 31/506; A61K 9/1694; A61K 9/2095; A61K 9/284; A61K 9/2013; A61K 9/2018; A61K 9/2054; A61K 9/2893

(57) ABSTRACT

A pharmaceutically acceptable composition comprising an effective amount of a dasatinib or a pharmaceutically acceptable salt thereof, and a binder, one or more diluent or mixture thereof, a disintegrant, a lubricant, wherein said composition is devoid of dasatinib hydrate or solvate or any crystalline polymorph, wherein the dasatinib or a pharmaceutically acceptable salt thereof, is present in an amorphous form.

### CROSS-REFERENCE TO RELATED APPLICATIONS

This application claims priority to Indian Provisional Application Serial No. 202021053103, filed Dec. 7, 2020, the entire disclosure of which is hereby incorporated by reference herein.

### FIELD OF THE INVENTION

The present invention relates to a pharmaceutically acceptable composition of dasatinib. More specifically, the present invention relates to a pharmaceutically acceptable composition of dasatinib, which is devoid of dasatinib hydrates, wherein the dasatinib or a pharmaceutically acceptable salt thereof, is present in an amorphous form.

### BACKGROUND OF THE INVENTION

Dasatinib, N-(2-chloro-6-methylphenyl)-2-[[6-[4-(2-hydroxyethyl)-1-piperazinyl]-2-methyl-4-pyrimidinyl]amino]-5-thiazolecarboxamide monohydrate a compound having the following chemical structure (formula I):

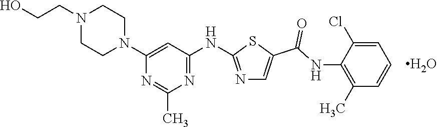

also known as BMS-354825, is a drug produced by Bristol-Myers Squibb and sold under the trade name Sprycel®. Dasatinib is an oral protein tyrosine kinase inhibitor, including Src Kinase, Bcr/Abl inhibitor, and is also known as a Src/Abl inhibitor, is useful in the treatment of oncological and immunologic diseases. Dasatinib is approved for use in patients with chronic myeloid leukemia (CML) after imatinib treatment, in newly diagnosed patients with chronic myeloid leukemia (CML) and Philadelphia chromosome-positive acute lymphoblastic leukemia (Ph+ ALL).

The recommended starting dosage of Sprycel® for chronic phase CML in adults is 100 mg administered orally once daily. The recommended starting dosage of Sprycel® for accelerated phase CML, myeloid or lymphoid blast phase CML, or Ph+ ALL in adults is 140 mg administered orally once daily.

Thus, in order to obtain sufficiently flexible dosing, individual dosage forms generally contain 20, 50 and 70 mg of dasatinib. Film coated tablets comprising the crystalline monohydrate of dasatinib are sold under the brand name Sprycel® (by Bristol Myers Squibb). This crystalline monohydrate of dasatinib is described in WO 2005/077945. Alternatively, the compound of formula (I) may exist in other crystalline forms, either as a neat compound or as a solvate.

The compound dasatinib and its preparation have been previously described in U.S. Pat. No. 6,596,746, issued Jul. 22, 2003. The use of the compound in the treatment of oncological and immunological disorders is described therein and in US Patent Publication No. US20040054186, published Mar. 18, 2004, which are both herein incorporated by reference.

WO 2006/121742 discloses conventional pharmaceutical formulations comprising dasatinib intended for oral administration. According to the examples, crystalline dasatinib monohydrate tablets are obtained by wet granulation. It also discloses pharmaceutical compositions having both intragranular and extragranular microcrystalline cellulose and having a non-reactive coating with polyethylene glycol as plasticizer.

Dasatinib is characterized as a low solubility/high permeability (BCS II) compound according to the Biopharmaceutics Classification System (BCS). The commercial dasatinib is a monohydrate of formula (I) with solubility of 8 μg/mL at 24° C. In this context, dissolution of dasatinib can potentially be rate-limiting for absorption.

US Patent Publication No. 20140343073 discloses a process for preparation of a stable amorphous form of dasatinib of formula (I). It also discloses the importance of micronized dasatinib to achieve the better particle size distribution in order to make the suitable formulation and also elaborates the different techniques for micronization.

WO 2017108605 A1 discloses a solid dispersion of dasatinib and a polymer, wherein the composition is obtained by dissolving dasatinib and the polymer in a solvent mixture comprising water, an alcohol and at least one molar equivalent acid with respect to dasatinib at a temperature ranging from 45 to 70° C., adding the resulting solution to a diluent, evaporating the solvent, mixing the resulting blend with further excipients finally compressing the final blend into tablets.

The physical properties of the crystalline form to that of an amorphous form are important in pharmaceutical processing. Each form may have advantage over others in, e.g., bioavailability, stability, or manufacturability.

The major problems that inhibit the use of the amorphous formulation are difficulty in the manufacturing and the poor chemical/physical stability. Notably, the bioavailability of the amorphous formulation with respect to the formulations containing crystalline forms is the most critical aspect for the formulators.

However, drugs that can exist in either amorphous or crystalline form tend to crystallize over time when present in amorphous state because the crystalline form of the drug is a lower-energy state than the amorphous form.

The solubility of amorphous forms is higher compared to the solubility of crystalline forms; thus, it would be desirable to have dasatinib available in amorphous form.

Accordingly, none of the above prior art documents discloses bioavailable amorphous dasatinib formulations, wherein such compositions have acceptable chemical stability, polymorphic stability & comparative dissolution as well as bioequivalence profile to that of Sprycel® tablets, thereby achieving target therapeutic effect, when administered to the patients.

Therefore, there is need in the prior art to develop a bioavailable amorphous dasatinib formulations having better purity, stability, storage, solubility, compressibility, homogeneity and flowability. This is a significant advancement in the state of the art.

### SUMMARY

Provided are pharmaceutical compositions of dasatinib.

Aspects of the present invention relate to providing a pharmaceutically acceptable composition comprising dasatinib, as an active agent, wherein the pharmaceutical composition comprises amorphous form of dasatinib and the process of preparation of the same.

Another aspect of the present invention is to provide a bioavailable composition of amorphous dasatinib.

Aspects of the present invention relate to provide an effective amount of a dasatinib or a pharmaceutically acceptable salt thereof, and a binder, one or more diluent or mixture thereof, a disintegrant, a lubricant, wherein said composition is devoid of dasatinib hydrate form.

### DETAILED DESCRIPTION OF THE INVENTION

As used herein, “a” or “an” means one or more unless otherwise specified.

The term “Patient” means an animal, preferably a mammal, more preferably human, in need of therapeutic intervention.

The term “Tyrosine-kinase inhibitor” is a pharmaceutical drug that inhibits tyrosine kinases. Tyrosine kinases are enzymes responsible for the activation of many proteins by signal transduction cascades. The proteins are activated by adding a phosphate group to the protein (phosphorylation), a step that Tyrosine-kinase inhibitors inhibit. Tyrosine-kinase inhibitors are typically used as anticancer drugs. For example, they have substantially improved outcomes in chronic myelogenous leukemia. There are several Tyrosine-kinase inhibitors were developed and approved by the FDA such as Imatinib, Gefitinib, Erlotinib, Sorafenib, Dasatinib, Sunitinib, Lapatinib, Nilotinib, Imatinib, Pazopanib, Crizotinib, Ruxolitinib, Vandetanib, Vemurafenib, Axitinib, Bosutinib, Cabozantinib, Pazopanib, Vismodegib, Ponatinib, Regorafenib, and Erlotinib.

The term “Composition” is intended to encompass a product comprising the specified ingredients in the specified amounts, as well as any product which results, directly or indirectly, from combinations of the specified ingredients in the specified amounts.

The term “% or percent by weight” is based on the total weight of the tablet.

The term “Pharmaceutically acceptable salt” refers includes, for example, a salt with an alkali metal such as lithium, sodium, potassium, etc.; a salt with an alkaline earth metal such as calcium, magnesium, etc.; a salt with zinc or aluminum; a salt with an organic base such as ammonium, choline, diethanolamine, lysine, ethylenediamine, t-butylamine, t-octylamine, tris(hydroxymethyl) aminomethane, N-methyl glucosamine, triethanolamine and dehydroabietylamine; a salt with an inorganic acid such as hydrochloric acid, hydrobromic acid, hydroiodic acid, sulfuric acid, nitric acid, phosphoric acid, etc.; or a salt with an organic acid such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, benzenesulfonic acid, etc.; or a salt with an acidic amino acid such as aspartic acid, glutamic acid, etc.

The term “solvate” is an aggregate that consists of a solute ion or molecule with one or more solvent molecules.

The term “hydrate” is a substance that contains water or its constituent elements.

The term “Polymorph” is one of several forms of crystal structure of a material displaying polymorphism.

The term “Amorphous” as used herein includes, but is not limited to, the substance lacking a crystalline structure.

The term “stable” and “stability” as used herein refers to both the physical form and the chemical purity of the amorphous dasatinib.

Another measure of chemical purity is defined by the content of other related impurities, by-products, or degradation products of the dasatinb (compound), which represents the morphology of the compound. The lower the content of total impurities as measured by HPLC, generally, by HPLC-UV, the higher the chemical purity of the compound. The total impurity content is measured after synthesis of the compound and compared with the measured total impurity content after storage for a designated period of time. Where the total impurity content does not significantly increase after storage, there has been no negative effect of storage on the chemical purity of the compound and the compound is stable for that designated period of time.

In one embodiment, at 0 days, the amorphous form of N-(2-chloro-6-methylphenyl)-2-[[6-[4-(2-hydroxyethyl)-1-piperazinyl]-2-methyl-4-pyrimidinyl]amino]-5-thiazolecarboxamide has a total impurity content of less than about 3% and, preferably, less than about 1%. In another embodiment, the amorphous form of N-(2-chloro-6-methylphenyl)-2-[[6-[4-(2-hydroxyethyl)-1-piperazinyl]-2-methyl-4-pyrimidinyl]amino]-5-thiazolecarboxamide has a total impurity content after storage for about 6 months at about 40° c. and about 75% RH of less than about 4% and, preferably, less than about 2%.

The term “Intra-granular portion A” as used herein includes, but is not limited to, a part or component of composition containing a mixture of one or more excipients which are further processed by one of the methods selected from the group of roller compaction or wet granulation or spray granulation or fluidized bed granulation methods to form a granule. Intra-granular portion A may contain an active ingredient i.e., dasatinib.

The term “Intra-granular portion B” as used herein includes, but is not limited to, a part or component of composition containing a mixture of one or more excipients which are further processed by one of the methods selected from the group of roller compaction or wet granulation or spray granulation or fluidized bed granulation methods to form a granule. Intra-granular portion B may contain an active ingredient i.e., dasatinib.

The term “Extra-granular or Extra-granular portion” as used herein includes, but is not limited to, a part or component of composition mixed or blended with granules prepared from Intra-granular portion A and/or Intra-granular portion B further subjected to compression to form the tablets.

One embodiment is directed to pharmaceutically acceptable composition comprising an effective amount of dasatinib or a pharmaceutically acceptable salt thereof, and a binder, one or more diluent or mixture thereof, a disintegrant, a lubricant, wherein said composition is devoid of dasatinib hydrate or solvate or any crystalline polymorph.

Another embodiment is directed to a pharmaceutically acceptable composition comprising an amorphous dasatinib as an active pharmaceutical ingredient, wherein said composition is devoid of dasatinib hydrate or solvate or any crystalline polymorph.

Another embodiment is directed to a pharmaceutically acceptable composition comprising an amorphous dasatinib, which has a comparative dissolution as well as bioequivalence profile to that of commercially available Sprycel® tablets, thereby achieving target therapeutic effect, when administered to the patients.

Another embodiment is directed to a pharmaceutically acceptable composition comprising dasatinib having better purity, stability, storage, solubility, compressibility, homogeneity and flowability.

Another embodiment is directed to a pharmaceutically acceptable composition comprising dasatinib, wherein the dasatinib is present in an amount from about 10% to 50% by weight of the total composition.

Another embodiment is directed to a pharmaceutically acceptable composition comprising dasatinib, wherein the dasatinib has d₉₀ particle size between about 10μ to about 250μ.

Another embodiment is directed to a pharmaceutically acceptable

composition comprising dasatinib and binder in an amount from about 2% to about 20% by weight of the total composition.

Another embodiment is directed to a pharmaceutically acceptable composition comprising dasatinib and hydroxyl propyl cellulose as a binder.

Another embodiment is directed to a pharmaceutically acceptable composition comprising dasatinib and hydroxyl propyl cellulose as a binder.

Another embodiment is directed to a pharmaceutically acceptable composition comprising dasatinib and hydroxyl propyl cellulose as a binder, may have a molecular weight between 50 to 1150 kDa.

Another embodiment is directed to a pharmaceutically acceptable composition comprising dasatinib and diluents.

Another embodiment is directed to a pharmaceutically acceptable composition comprising dasatinib and diluents such as microcrystalline cellulose and/or lactose or mixture thereof.

Another embodiment is directed to a pharmaceutically acceptable composition comprising dasatinib and diluent such as microcrystalline cellulose, which may present in intra-granular portion A or intra-granular portion B or in both intra-granular portion A and intra-granular portion B.

Another embodiment is directed to a pharmaceutically acceptable composition comprising dasatinib and diluent such as lactose, which may present in intra-granular portion A or intra-granular portion B or in both intra-granular portion A and intra-granular portion B.

Another embodiment is directed to a pharmaceutically acceptable composition comprising dasatinib and diluents such as microcrystalline cellulose and/or lactose or mixture thereof, wherein the ratio of lactose to microcrystalline cellulose is ranges from about 1:0.1 to about 3:10 by weight of total composition.

Another embodiment is directed to a pharmaceutically acceptable composition comprising dasatinib and disintegrant, wherein the disintegrant is crosscarmellose sodium.

According to the invention a pharmaceutically acceptable composition comprising dasatinib or a pharmaceutically acceptable salt thereof, and a binder, one or more diluent or mixture thereof, a disintegrant, a lubricant.

According to the invention a pharmaceutically acceptable composition comprising one or more binder/s selected from the group of cellulose, cellulose derivatives, gelatin, glucose, lactose, cellulose derivatives-methyl cellulose, ethyl cellulose, hydroxy propylmethyl cellulose, hydroxy propyl cellulose, starch, poly vinyl pyrrolidone (Povidone), sodium alginate, carboxymethylcellulose, acacia and pregelatinized maize starch. In certain embodiment, hydroxy propyl cellulose is the most preferred binder.

In yet another aspect of the invention, different grades of hydroxy propyl cellulose used as a binder. There are multiple molecular weight grades of hydroxy propyl cellulose are commercially available such as Klucel™. These commercially available grades of hydroxy propyl cellulose are differentiated on their molecular weights. These commercially available grades of hydroxyl propyl cellulose include EXF, LXF, JXF, GXF, MXF and HXF having an average weight molecular weight of 80, 95, 140, 370, 850 and 1150 (kDa) respectively.

Another embodiment is directed to a pharmaceutically acceptable composition comprising at least one grade of hydroxyl propyl cellulose include EXF, LXF, JXF, GXF, MXF and HXF having an average molecular weight of 80, 95, 140, 370, 850 and 1150 (kDa).

Another embodiment is directed to a pharmaceutically acceptable composition comprising dasatinb and binder is present in an amount from about 2% to about 20% by weight of total composition. Preferably, the binder is present in an amount from about 2% to about 10% by weight of total composition. More preferably, the binder is present in an amount from about 2% to about 8% by weight of total composition. Most preferably, the binder is present in an amount from about 4% to about 8% by weight of total composition.

According to the invention, a pharmaceutically acceptable composition comprising one or more diluent/s selected from the group of lactose, anhydrous lactose, spray dried lactose, lactose monohydrate, micro crystalline cellulose (Avicel® PH 101 and PH 102), cellulose powder, dibasic calcium phosphate anhydrous, dibasic calcium phosphate dihydrate, calcium phosphate, calcium sulfate anhydrous, calcium carbonate, calcium lactate, glycerin palmitostearate, kaolin, lactitol, starch derivates, corn starch, cellulose derivates, magnesium carbonate, maltitol, maltodextrin, mannitol, polydextrose, povidone, sorbitol, maize starch, pregelatinized starch, trehalose, sucrose and xylitol.

Another embodiment is directed to a pharmaceutically acceptable composition comprising dasatinb and microcrystalline cellulose and/or lactose or mixture thereof. Preferably, the diluents are microcrystalline cellulose and/or lactose.

Another embodiment is directed to a pharmaceutically acceptable composition comprising dasatinb and microcrystalline cellulose and/or lactose are present in both intra-granular portion A as well as intra-granular portion B.

Another embodiment is directed to a pharmaceutically acceptable composition comprising dasatinib and lactose, wherein the lactose has some remarkable impact on the release of dasatinib from the composition. Lactose will soluble at a faster rate than that of Dasatinib leads to slow down the Dasatinib release from the composition.

Another embodiment is directed to a pharmaceutically acceptable composition comprising dasatinb and lactose, wherein the lactose is present in both intra-granular portion A as well as intra-granular portion B.

Another embodiment is directed to a pharmaceutically acceptable composition comprising dasatinb and lactose, wherein the lactose is present in an intra-granular portion A.

Another embodiment is directed to a pharmaceutically acceptable composition comprising dasatinb and lactose, wherein the lactose is present in an intra-granular portion B.

Another embodiment is directed to a pharmaceutically acceptable composition comprising dasatinb and microcrystalline cellulose, wherein the microcrystalline cellulose is present in both intra-granular portion A as well as intra-granular portion B.

Another embodiment is directed to a pharmaceutically acceptable composition comprising dasatinb and microcrystalline cellulose, wherein the microcrystalline cellulose is present in an intra-granular portion A.

Another embodiment is directed to a pharmaceutically acceptable composition comprising dasatinib and microcrystalline cellulose, wherein the microcrystalline cellulose is present in an intra-granular portion B.

Another embodiment is directed to a pharmaceutically acceptable composition comprising dasatinb and diluents are present in an amount of from about 1% to about 90% by weight of the total composition. Preferably, the diluents are present in an amount of from about 5% to about 80% by weight of the total composition. More preferably, the diluents are present in an amount of from about 25% to about 75% by weight of the total composition. Most preferably, the diluents are present in an amount of from about 40% to about 70% by weight of the total composition.

Another embodiment is directed to a pharmaceutically acceptable composition having ratio of lactose to microcrystalline cellulose is ranges from about 1:0.1 to about 4:10 by weight of total composition.

According to the invention, a pharmaceutically acceptable composition comprising disintegrant selected from the group of crosslinked cellulose derivatives, croscarmellose sodium (crosslinked carboxy-methylcellulose sodium or Ac-Di-Sol), microcrystalline cellulose, alginates such as crosslinked alginic acid, ion-exchange resins, hydrous aluminium silicate, cross-linked polyplasdone, sodium starch glycolate, crosslinked polyvinylpyrrolidone, soy polysaccharides, pregelatinized starch and calcium silicate.

Another embodiment is directed to a pharmaceutically acceptable composition comprising dasatinib and disintegrant is present in an amount of from about 0.1% to about 10% by weight of the total composition. Preferably, the disintegrant is present in an amount of from about 0.5% to about 8% by weight of the total composition. More preferably, the disintegrant is present in an amount of from about 1% to about 6% by weight of the total composition. Most preferably, the disintegrant is present in an amount of from about 2% to about 5% by weight of the total composition.

Another embodiment is directed to a pharmaceutically acceptable composition comprising dasatinb and disintegrant, wherein the disintegrant is present in both intra-granular portion A and intra-granular portion B.

Another embodiment is directed to a pharmaceutically acceptable composition comprising dasatinb and disintegrant, wherein the disintegrant is present in intra-granular portion A.

Another embodiment is directed to a pharmaceutically acceptable composition comprising dasatinb and disintegrant, wherein the disintegrant is present in intra-granular portion B.

According to the invention, a pharmaceutically acceptable composition has disintegration time between 5 minutes to 30 minutes. Preferably, a pharmaceutically acceptable composition has disintegration time between 10 minutes to 30 minutes. More preferably, a pharmaceutically acceptable composition has disintegration time between 15 minutes to 30 minutes. Most preferably, a pharmaceutically acceptable composition has disintegration time between 15 minutes to 25 minutes.

According to the invention, a pharmaceutically acceptable composition comprising lubricant selected from the group of stearic acid derivatives, stearates, magnesium stearate, sodium stearyl fumarate, stearyl alcohol, talc, ethylene glycol stearates and glyceryl behenate.

Another embodiment is directed to a pharmaceutically acceptable composition comprising dasatinib and lubricant is present in an amount of from about 0.25% to about 5.0% by weight of the total composition.

Another embodiment is directed to a pharmaceutically acceptable composition comprising dasatinib is prepared by a process comprising the following steps:

(a) sifting of an intragranular excipients and blended in a suitable blender;

(b) lubrication of blended granules of step (a);

(c) compaction of the lubricated granules of step (b) using roller compactor to form flakes followed by milling;

(d) sifting of an extra-granular material;

(e) blending and lubricating material of step (d) and step (c);

(f) compaction of the lubricated granules of step (e) using compression machine;

(d) coating the tablet.

Another embodiment is directed toward a pharmaceutically acceptable composition comprising dasatinib is prepared by a process comprising the following steps:

(a) sifting of an intra-granular portion A excipients and blended in a suitable blender;

(b) lubrication of blended granules of step (a);

(c) compaction of the lubricated granules of step (b) using roller compactor to form flakes followed by milling;

(d) sifting of an intra-granular portion B excipients followed by blending;

(e) lubrication of blended granules of step (d);

(f) compaction of the lubricated granules of step (e) using roller compactor to form flakes followed by milling;

(g) blending and lubricating granules from the step (c) and step (f);

(h) compaction of the blended granules of step (g) to form a tablet;

(i) coating the tablet.

Another embodiment is directed to a pharmaceutically acceptable composition comprising dasatinib is prepared by a process comprising the following steps:

(a) sifting of an excipient and blended in a suitable blender;

(b) lubrication of blended granules of step (a);

(c) compaction of the lubricated granules of step (b) using compression machine;

(d) coating the tablet.

All numbers expressing quantities of ingredients, properties such as molecular weight, viscosity, amount and so forth that are preceded by the word “about” are to be understood as only approximations so that slight variations above and below the stated number may be used to achieve substantially the same results as the stated number. Accordingly, unless indicated to the contrary, numerical parameters preceded by the word “about” are approximations that may vary depending upon the desired properties sought to be obtained by the present invention.

It is to be understood that each of the variously stated ranges is intended to be continuous so as to include each numerical parameter between the stated minimum and maximum value of each range.

The invention having been described in detail; the following examples are presented to show specific embodiments thereof. It will be understood the examples are given for illustration purposes only and not by way of limitation.

### Example 1

| | | |
|---|---|---|
| | Amount | |
| Ingredient | (mg) | % (w/w) |

Intra-granular

| | | |
|---|---|---|
| Dasatinib (Amorphous) | 20.00 | 24.27 |
| Microcrystalline cellulose | 15.80 | 19.17 |
| Hydroxy propyl cellulose | 4.00 | 4.85 |
| Magnesium stearate | 1.60 | 1.94 |

Extra-granular

| | | |
|---|---|---|
| Lactose anhydrous | 33.00 | 40.04 |
| Croscarmellose sodium | 4.00 | 4.85 |
| Hydroxy propyl cellulose | 0.80 | 0.97 |
| Magnesium stearate | 0.80 | 0.97 |
| Core tablet weight | 80.00 | 97.09 |
| Opadry white 03B28796 | 2.40 | 2.91 |
| Coated tablet weight | 82.40 | 100.00 |

### Example 2

| | | |
|---|---|---|
| | Amount | |
| Ingredient | (mg) | % (w/w) |

Intra-granular

| | | |
|---|---|---|
| Dasatinib (Amorphous) | 20.00 | 24.27 |
| Dibasic calcium phosphate anhydrous | 8.00 | 9.70 |
| Hydroxy propyl cellulose | 4.00 | 4.85 |
| Magnesium stearate | 1.60 | 1.94 |

Extra-granular

| | | |
|---|---|---|
| Lactose anhydrous | 27.00 | 32.76 |
| Mannitol | 15.80 | 19.17 |
| Croscarmellose sodium | 2.00 | 2.42 |
| Hydroxy propyl cellulose | 0.80 | 0.97 |
| Magnesium stearate | 0.80 | 0.97 |
| Core tablet weight | 80.00 | 97.09 |
| Opadry white 03B28796 | 2.40 | 2.91 |
| Coated tablet weight | 82.40 | 100.00 |

### Example 3

| | | |
|---|---|---|
| | Amount | |
| Ingredient | (mg) | % (w/w) |

Intra-granular portion A

| | | |
|---|---|---|
| Dasatinib (Amorphous) | 20.00 | 22.88 |
| Microcrystalline cellulose | 27.00 | 30.89 |
| Hydroxy propyl cellulose | 7.00 | 8.00 |
| Magnesium stearate | 1.60 | 1.83 |

Intra-granular portion B

| | | |
|---|---|---|
| Microcrystalline Cellulose | 23.80 | 27.23 |
| Croscarmellose sodium | 4.00 | 4.57 |
| Hydroxy propyl cellulose | 0.80 | 0.91 |
| Magnesium stearate | 0.80 | 0.91 |
| Core tablet weight | 85.00 | 97.25 |
| Opadry white 03B28796 | 2.40 | 2.74 |
| Coated tablet weight | 87.40 | 100.00 |

### Example 4

| | | |
|---|---|---|
| | Amount | |
| Ingredient | (mg) | % (w/w) |

Intra-granular

| | | |
|---|---|---|
| Dasatinib (Amorphous) | 20.00 | 24.27 |
| Mannitol | 8.00 | 9.70 |
| Hydroxy propyl cellulose | 4.00 | 4.85 |
| Magnesium stearate | 1.60 | 1.94 |

Extra-granular

| | | |
|---|---|---|
| Microcrystalline Cellulose | 42.8 | 51.94 |
| Croscarmellose sodium | 2.00 | 2.42 |
| Hydroxy propyl cellulose | 0.80 | 0.97 |
| Magnesium stearate | 0.80 | 0.97 |
| Core tablet weight | 80.00 | 97.09 |
| Opadry white 03B28796 | 2.40 | 2.91 |
| Coated tablet weight | 82.40 | 100.00 |

### Example 5

| | | |
|---|---|---|
| | Amount | |
| Ingredient | (mg) | % (w/w) |

Intra-granular

| | | |
|---|---|---|
| Dasatinib (Amorphous) | 20.00 | 24.27 |
| Sorbitol | 8.00 | 9.70 |
| Hydroxy propyl cellulose | 4.00 | 4.85 |
| Magnesium stearate | 1.60 | 1.94 |

Extra-granular

| | | |
|---|---|---|
| Sorbitol | 17.00 | 20.63 |
| Microcrystalline Cellulose | 25.80 | 31.31 |
| Croscarmellose sodium | 2.00 | 2.42 |
| Hydroxy propyl cellulose | 0.80 | 0.97 |
| Magnesium stearate | 0.80 | 0.97 |
| Core tablet weight | 80.00 | 97.08 |
| Opadry white 03B28796 | 2.40 | 2.91 |
| Coated tablet weight | 82.40 | 100.00 |

The above examples were prepared using the following procedure:

(a) sifting of an intra-granular excipient and blended in a suitable blender;

(b) lubrication of blended granules of step (a);

(c) compaction of the lubricated granules of step (b) using roller compactor with integrated milling machine;

(d) sifting of an extra-granular excipient and blended with lubricated granules of step (c) in a suitable blender;

(e) lubrication of blended granules;

(f) compaction of the blended granules of step (e) to form a tablet;

(g) coating the tablet.

### Example 6

| | | |
|---|---|---|
| | Amount | |
| Ingredient | (mg) | % (w/w) |

Intra-granular portion A

| | | |
|---|---|---|
| Dasatinib (Amorphous) | 20.00 | 23.98 |
| Dibasic calcium phosphate anhydrous | 8.00 | 9.59 |
| Microcrystalline Cellulose | 28.00 | 33.57 |
| Hydroxy propyl cellulose | 4.00 | 4.79 |
| Magnesium stearate | 1.60 | 1.91 |

Intra-granular portion B

| | | |
|---|---|---|
| Microcrystalline Cellulose | 15.00 | 17.98 |
| Croscarmellose sodium | 2.00 | 2.39 |
| Hydroxy propyl cellulose | 0.80 | 0.95 |
| Magnesium stearate | 0.80 | 0.95 |

Extra-granular portion

| | | |
|---|---|---|
| Magnesium stearate | 0.80 | 0.95 |
| Core tablet weight | 81.00 | 97.12 |
| Opadry white 03B28796 | 2.40 | 2.87 |
| Coated tablet weight | 83.40 | 100.00 |

### Example 7

| | | |
|---|---|---|
| | Amount | |
| Ingredient | (mg) | % (w/w) |

Intra-granular portion A

| | | |
|---|---|---|
| Dasatinib (Amorphous) | 20.00 | 24.27 |
| Hydroxy propyl cellulose | 4.00 | 4.85 |
| Lactose anhydrous | 8.00 | 9.70 |
| Magnesium stearate | 1.60 | 1.94 |

Intra-granular portion B

| | | |
|---|---|---|
| Lactose anhydrous | 27.00 | 32.76 |
| Microcrystalline Cellulose | 15.80 | 19.17 |
| Croscarmellose sodium | 2.00 | 2.42 |
| Hydroxy propyl cellulose | 0.80 | 0.97 |
| Magnesium stearate | 0.40 | 0.48 |

Extra-granular

| | | |
|---|---|---|
| Magnesium stearate | 0.40 | 0.48 |
| Core tablet weight | 80.00 | 97.09 |
| Opadry white 03B28796 | 2.40 | 2.91 |
| Coated tablet weight | 82.40 | 100.00 |

### Example 8

| | | |
|---|---|---|
| | Amount | |
| Ingredient | (mg) | % (w/w) |

Intra-granular portion A

| | | |
|---|---|---|
| Dasatinib (Amorphous) | 20.00 | 24.03 |
| Microcrystalline cellulose | 27.00 | 32.45 |
| Hydroxy propyl cellulose | 4.00 | 4.80 |
| Magnesium stearate | 1.60 | 1.92 |

Intra-granular portion B

| | | |
|---|---|---|
| Dibasic calcium phosphate anhydrous | 8.00 | 9.61 |
| Microcrystalline Cellulose | 15.80 | 18.99 |
| Croscarmellose sodium | 2.00 | 2.40 |
| Hydroxy propyl cellulose | 0.80 | 0.96 |
| Magnesium stearate | 0.80 | 0.96 |

Extra-granular

| | | |
|---|---|---|
| Magnesium stearate | 0.80 | 0.96 |
| Core tablet weight | 80.80 | 97.11 |
| Opadry white 03B28796 | 2.40 | 2.88 |
| Coated tablet weight | 83.20 | 100.00 |

### Example 9

| | | |
|---|---|---|
| | Amount | |
| Ingredient | (mg) | % (w/w) |

Intra-granular portion A

| | | |
|---|---|---|
| Dasatinib (Amorphous) | 20.00 | 24.03 |
| Hydroxy propyl cellulose | 6.00 | 7.21 |
| Lactose anhydrous | 8.00 | 9.61 |
| Magnesium stearate | 1.60 | 1.92 |

Intra-granular portion B

| | | |
|---|---|---|
| Lactose anhydrous | 20.00 | 24.03 |
| Microcrystalline Cellulose | 20.80 | 25.00 |
| Croscarmellose sodium | 2.00 | 2.40 |
| Hydroxy propyl cellulose | 0.80 | 0.96 |
| Magnesium stearate | 0.80 | 0.96 |

Extra-granular

| | | |
|---|---|---|
| Magnesium stearate | 0.80 | 0.96 |
| Core tablet weight | 80.80 | 97.11 |
| Opadry white 03B28796 | 2.40 | 2.88 |
| Coated tablet weight | 83.20 | 100.00 |

### Example 10

| | | |
|---|---|---|
| | Amount | |
| Ingredient | (mg) | % (w/w) |

Intra-granular portion A

| | | |
|---|---|---|
| Dasatinib (Amorphous) | 20.00 | 24.03 |
| Hydroxy propyl cellulose | 4.00 | 4.80 |
| Lactose anhydrous | 20.00 | 24.03 |
| Magnesium stearate | 1.60 | 1.92 |

Intra-granular portion B

| | | |
|---|---|---|
| Lactose anhydrous | 20.00 | 24.03 |
| Microcrystalline Cellulose | 10.80 | 12.98 |
| Croscarmellose sodium | 2.00 | 2.40 |
| Hydroxy propyl cellulose | 0.80 | 0.96 |
| Magnesium stearate | 0.80 | 0.96 |

Extra-granular

| | | |
|---|---|---|
| Magnesium stearate | 0.80 | 0.96 |
| Core tablet weight | 80.80 | 97.11 |
| Opadry white 03B28796 | 2.40 | 2.91 |
| Coated tablet weight | 83.20 | 100.00 |

The above examples were prepared using the following procedure:

(a) sifting of an intra-granular portion A excipient and blended in a suitable blender;

(b) lubrication of blended granules of step (a);

(c) compaction of the lubricated granules of step (b) using roller compactor to form flakes followed by milling;

(d) sifting of an intra-granular portion B excipient followed by blending;

(e) lubrication of blended granules of step (d);

(f) compaction of the lubricated granules of step (e) using roller compactor to form flakes followed by milling;

(g) blending and lubricating granules from the step (c) and step (f);

(h) compaction of the blended granules of step (g) to form a tablet;

(i) coating the tablet.

### Example 11

| | | |
|---|---|---|
| | Amount | |
| Ingredient | (mg) | % (w/w) |

Intra-granular portion A

| | | |
|---|---|---|
| Lactose anhydrous | 26.59 | 32.27 |
| Microcrystalline Cellulose | 15.80 | 19.17 |
| Croscarmellose sodium | 2.00 | 2.43 |
| Hydroxy propyl cellulose | 0.80 | 0.97 |
| Magnesium stearate | 0.40 | 0.49 |

Intra-granular portion B

| | | |
|---|---|---|
| Dasatinib (Amorphous) | 20.00 | 24.27 |
| Hydroxy propyl cellulose | 4.00 | 4.85 |
| Lactose anhydrous | 8.408 | 10.20 |
| Magnesium stearate | 1.60 | 1.94 |

Extra-granular

| | | |
|---|---|---|
| Magnesium stearate | 0.40 | 0.49 |
| Core tablet weight | 80.00 | 97.09 |
| Opadry white 03B28796 | 2.40 | 2.91 |
| Coated tablet weight | 82.40 | 100.00 |

The above examples were prepared using the following procedure:

(a) sifting of an intra-granular portion A excipient and blended in a suitable blender;

(b) lubrication of blended granules of step (a);

(c) compaction of the lubricated granules of step (b) using roller compactor to form flakes followed by milling;

(d) sifting of an intra-granular portion B excipient followed by blending;

(e) lubrication of blended granules of step (d);

(f) compaction of the lubricated granules of step (e) using roller compactor to form flakes followed by milling;

(g) blending and lubricating granules from the step (c) and step (1);

(h) compaction of the blended granules of step (g) to form a tablet;

(i) coating the tablet.

Dissolution Study:

The dissolution study was conducted for the compositions of the present invention comprising amorphous dasatinib tablets 100 mg strength in comparison with the Sprycel® (dasatinib) 100 mg tablets (Marketed by Bristol Myers Squibb) in OGD media (pH 4.0 acetate buffer+1% Triton X 100, 1000 ml) at 60 rpm using USP type II Paddle apparatus. The samples were analyzed using HPLC technique.

| Time Interval | Percent (%) amount of drug released | |
|---|---|---|
| (mins) | Example 1 | Example 2 |
| 20 | 45 | 46 |
| 30 | 73 | 76 |
| 45 | 101 | 96 |
| 60 | 103 | 96 |

Results of dissolution study shows that compositions of the present invention comprising amorphous dasatinib showed no significant difference from the Sprycel® tablets (Marketed by Bristol Myers Squibb). Dissolution studies were also conducted at predefined time intervals for on-stability samples of compositions of the present invention comprising amorphous dasatinib.

Bioequivalence Study:

An open label, balanced, randomized, two-treatment, two-period, two-sequence, single oral dose, crossover, oral bioequivalence study of dasatinib Tablets 100 mg in comparison with Sprycel® (dasatinib) tablets 100 mg was performed in healthy, adult, human subjects under fasting and fed conditions.

The outcome of the bioequivalence study for compositions of the present invention comprising amorphous dasatinib under fed and fasting state comprising amorphous dasatinib complies to pre-defined bioequivalence criteria for Cₘₐₓ, AUC₀₋ₜ and AUC_{0-∞}.

Stability Study:

Stability studies were conducted, according to the ICH guidelines, for 6 months at accelerated condition (40° C./75% RH) and at room temperature. Based on the parameters such as physical appearance and chemical impurity, the pharmaceutically acceptable compositions of the present invention were found to be stable.

At 0 days, the amorphous form of N-(2-chloro-6-methylphenyl)-2-[[6-[4-(2-hydroxyethyl)-1-piperazinyl]-2-methyl-4-pyrimidinyl]amino]-5-thiazolecarboxamide has a total impurity content of less than about 3% and, preferably, less than about 1%. In another embodiment, the amorphous form of N-(2-chloro-6-methylphenyl)-2-[[6-[4-(2-hydroxyethyl)-1-piperazinyl]-2-methyl-4-pyrimidinyl]amino]-5-thiazolecarboxamide has a total impurity content after storage for about 6 months at about 40° c. and about 75% RH of less than about 4% and preferably, less than about 2%.

What is claimed is:

1. A pharmaceutically acceptable composition comprising an effective amount of a dasatinib or a pharmaceutically acceptable salt thereof, and a binder, one or more diluent or mixture thereof, a disintegrant, a lubricant, wherein said composition is devoid of dasatinib hydrate or solvate or any crystalline polymorph.

2. A pharmaceutically acceptable composition as claimed in claim 1, wherein the dasatinib or a pharmaceutically acceptable salt thereof, is present in an amorphous form.

3. A pharmaceutically acceptable composition as claimed in claim 1, wherein the binder is hydroxy propyl cellulose; and wherein the binder is present in an amount from about 2% to about 10% by weight of total composition.

4. A pharmaceutically acceptable composition as claimed in claim 1, wherein the diluent is lactose, microcrystalline cellulose, or combination thereof; wherein the diluent is present in an amount from about 1% to about 90% by weight of the total composition.

5. A pharmaceutically acceptable composition as claimed in claim 4, wherein the ratio of lactose to microcrystalline cellulose is ranges from about 1:0.1 to about 4:10 by weight of total composition.

6. A pharmaceutically acceptable composition as claimed in claim 4, wherein the lactose is present in an amount from about 10% to about 70% by weight of the total composition.

7. A pharmaceutically acceptable composition as claimed in claim 5, wherein the lactose is present in both intra-granular A and intra-granular portion B.

8. A pharmaceutically acceptable composition as claimed in claim 1, wherein the disintegrant is croscarmellose sodium.

9. A pharmaceutically acceptable composition as claimed in claim 1, wherein said composition has disintegration time between 5 minutes to 30 minutes.

10. A pharmaceutically acceptable composition as claimed in claim 1, prepared by a process comprising the following steps:
(a) sifting of an intra-granular portion A excipient and blended in a suitable blender;
(b) lubrication of blended granules of step (a);
(c) compaction of the lubricated granules of step (b) using roller compactor to form flakes followed by milling;
(d) sifting of an intra-granular portion B excipient followed by blending;
(e) lubrication of blended granules of step (d);
(f) compaction of the lubricated granules of step (e) using roller compactor to form flakes followed by milling;
(g) blending and lubricating granules from the step (c) and step (f);
(h) compaction of the blended granules of step (g) to form a tablet;
(i) coating the tablet.
